(19) United States
(12) Patent Application Publication
(10) Pub. No.: US 2010/0136696 A1
(43) Pub. Date: Jun. 3, 2010

(54) APPARATUS AND METHOD FOR THE ENHANCEMENT OF CONTAMINANT DETECTION

(75) Inventors: Daniel L. Schaefer, Wichita, KS (US); Rudy Steiner, Dodge City, KS (US)

Correspondence Address: CARGILL, INCORPORATED, P.O. Box 5624, MINNEAPOLIS, MN, 55440-5624, US

(73) Assignee: CARGILL INCORPORATED, Wayzata, MN (US)

(21) Appl. No.: 12/521,726

(22) PCT Filed: Dec. 27, 2007
(86) PCT No.: PCT/US07/26353

§ 371 Date: Nov. 23, 2009

Related U.S. Application Data
(60) Provisional application No. 60/878,203, filed on Dec. 29, 2006.

(51) Int. Cl. G01N 33/02; G01N 21/00; H05G 1/00

(52) U.S. Cl. 436/20; 422/68.1; 422/82.05; 422/104; 378/208

(57) ABSTRACT

An apparatus and method for detecting contaminants in a food product having an irregular shape is described. The apparatus comprises a contaminant detection device constructed and arranged to detect contaminants in a food product when the food product enters a zone of operation relative to the contaminant detection device. A conveyor belt is electrically coupled to the contaminant detector. The contour filler is operably connected to the conveyor bell and contaminant detector, and positions the food product within the zone of operation. The contour filler may retain the food product and fill the cross sectional area of the zone of operation to enhance contaminant detection. The contour filler may rotate the food product relative to the contaminant detector.

### PRIORITY CLAIM

This application claims the benefit of U.S. Provisional Application having Ser. No. 60/878,203, filed Dec. 29, 2006, entitled “CONTOUR FILLER FOR THE ENHANCEMENT OF CONTAMINANT DETECTION,” which application is incorporated herein by reference in its entirety.

### FIELD OF THE INVENTION

An apparatus and method for enhancement of contaminant detection is described. More specifically, an apparatus and method for the enhancement of contaminant detection in round, spherical, or cylindrical products by contaminant detection devices, such as x-ray equipment, is described.

### BACKGROUND

X-ray equipment is currently in use to inspect products. While x-ray equipment is useful, there is a particular problem with its use for round, spherical or cylindrical products, as the x-ray equipment may fail to detect contaminant matter in products of such shape, or other irregular shapes. Currently, x-ray equipment has limitations in detecting contaminants whenever they are present at the outside edge of a round, spherical or cylindrical product.

The limitations of x-ray equipment in detecting contaminants at the outside edge of a round, spherical or cylindrical product are due to the fact that the mass of the product that x-rays travel through at its outside edge is a lot less than the mass they travel through at its center or interior portion (see FIG. 1). FIG. 1 illustrates prior art x-ray equipment, including an x-ray source 20 and x-ray detector 22, with a round, spherical, or cylindrical product 10 located between the source 20 and detector 22, and being exposed to x-rays 24. The product 10 has less mass at the outside edges 12, 14 through which the x-rays 24 travel, and is, therefore, being exposed to higher energy levels at the edges 12, 14 than in the more interior portions of the product 10. The higher energy level at the outside edges 12, 14 is sufficient to travel through a contaminant 16 that is present at or near that edge 12, thus causing the x-ray not to detect the contaminant 16. This is of particular significance, for example, when the product is a meat chub or sausage chub.

Thus, a needs exists for a way to detect the presence of contaminants in the edge portions of round, spherical or cylindrical shaped products by using x-ray equipment, or other similar contaminant detection devices.

### SUMMARY

Accordingly, an apparatus and method for detecting contamination present in irregularly shaped products, such as round, spherical, or cylindrical food products, for example, is disclosed.

One aspect is an apparatus for detecting contaminants in a food product having an irregular shape. The apparatus may comprise a contaminant detection device constructed and arranged to detect contaminants in a food product when the food product enters a predetermined zone of operation relative to the contaminant detection device; a conveyor belt electrically coupled to the contaminant detection device for conveying the food product to the zone of operation; and a contour filler electrically coupled to the conveyor belt and the contaminant detection device, the contour filler being constructed and arranged to retain and position the food product within the zone of operation. The zone of operation of the contaminant detection device may have a uniform cross sectional area; and the contour tiller may be constructed and arranged to position the food product in the zone of operation, such that the food product fills a first portion of the cross sectional area of the zone of operation, and the contour filler tills a second remaining portion of the cross sectional area of the zone of operation. The contaminant detection device may be selected from the group consisting of an x-ray source, a source of ultrasound energy, a source of near infrared energy, a source of electron beam energy, a source of ultraviolet light, a source of visible light, a source of microwave, and a source of gamma radiation. The contaminant detection device may be an x-ray source, and zone of operation may correspond to an x-ray beam emitted there from.

The contour filler may comprise an exterior portion made of a soft, compliant material, filled with a fluid. The exterior of the contour filler may include a recessed portion shaped to accommodate the food product. The exterior portion may be made of a material selected from the group consisting of a plastic, rubber or elastomeric material, and the contour filler is filled with a fluid material selected from the group consisting of a liquid, gel or soft material. The contour filler may comprise a bag or bladder made of an elastomeric material, encasing a tilling material selected from the group consisting of water, foam or silicone. The contour filler may comprise a flexible semi-hemispherical conveyor belt. The semi-hemispherical conveyor bell may comprise a plurality of flexible segments constructed and arranged to retain the food product. The contour filler may comprise a pair of guide rails constructed and arranged to hold the food product in an interior portion thereof, the pair further comprising a first adjustable semi-hemispherical guide rail and a second adjustable semi-hemispherical guide rail. The first and second guide rails may each further comprise a lateral adjustable arm, and may be adjustable as to their position by the lateral arms. The guide rails may comprise a material selected from the group consisting of UMHW polyethylene, and TIVAR®. The contour filler may comprise a retaining means and a means for rotating the food product within the zone of operation in an axis perpendicular thereto.

A second aspect is a method for detecting contaminants in a product having an irregular shape. The method may comprise the steps of: a) providing a round, spherical or cylindrical object; b) providing a contaminant detection device operable within a predetermined zone of operation having a uniform cross sectional area, the device being constructed and arranged to detect contaminants in a round, spherical, or cylindrical object within the zone of operation; c) initiating a contaminant detection process upon detecting the object within the zone of operation; and, d) initiating a contour filling procedure to enhance contaminant detection by the contaminant detection device. The contour filling procedure may include the step of providing a contour filler constructed and arranged to retain and position the object within the zone of operation, such that the object fills a first portion of the cross sectional area of the zone of operation and the contour filler operates to fill a second remaining portion of the cross sectional area of the zone of operation of the contaminant detection device. The contour filling procedure may include the step of providing means for rotating the object relative to the contaminant detection device. The contaminant detection device may be selected from the group consisting of an x-ray source, a source of ultrasound energy, a source of near infrared energy, a source of electron beam energy, a source of ultraviolet light, a source of visible light, a source of microwave, and a source of gamma radiation.

A third aspect is an apparatus for enhancement of contaminant detection in a product having an irregular shape using a contaminant detection device, the apparatus comprising a contour tiller comprising a recessed portion to accommodate the irregular shape of the product, wherein when the product is in the recessed portion, together the contour filler and the product provide a cross sectional area in which contaminants may be identified by the contaminant detection device. The cross sectional area of the contour filler and product may together be substantially uniform. The contour fuller may comprise two portions that combine to provide the recessed portion. The contaminant detection device may be selected from the group consisting of an x-ray source, a source of ultrasound energy, a source of near infrared energy, a source of electron beam energy, a source of ultraviolet light, a source of visible light, a source of microwave, and a source of gamma radiation. The contour filler may comprise an exterior portion made of a soft, compliant material, filled with a fluid. The contour filler may comprise a conveyor belt. The cross sectional shape may be selected from the group consisting of a square and a rectangle.

### BRIEF DESCRIPTION OF THE DRAWINGS

FIG. 1 is an illustration of prior art x-ray equipment with x-rays from the equipment traveling through a product having a circular cross-sectional shape;

FIG. 2 shows an embodiment of an apparatus in accordance with the invention;

FIG. 3 shows an alternative embodiment of a device in accordance with the invention;

FIG. 4 shows another alternative embodiment of an apparatus in accordance with the invention; and

FIG. 5 shows a method for detecting contamination in round, spherical, or cylindrical products.

### DETAILED DESCRIPTION OF THE PREFERRED EMBODIMENTS

It may be desirable to artificially increase a product's mass around its outside edge in order to increase contaminant detection rates using a contaminant detector, such as x-ray equipment.

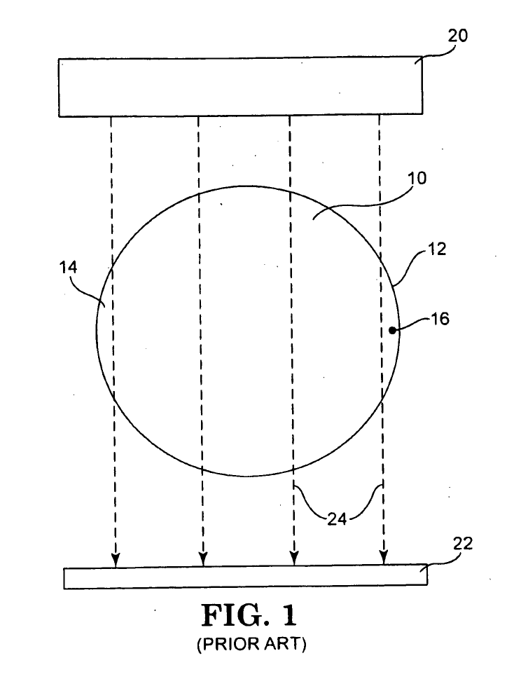

Contaminants present at the outside edge of round, spherical, or cylindrical products are hard to detect using current contaminant detector devices. For example, a chub of a ground food product may be provided in a cylindrical package in pound, 2 pound, three pound, five pound, ten pound and 22 pound sizes. When such a product package is exposed to an x-ray device, the x-rays pass through the cross-sectional area of product 10, as shown at FIG. 1. X-rays pass through the product 10 at or near the edges 12, 14 more easily because of the lower amount of mass of product 10 at the edges 12, 14 as compared to the interior portion of the product 10. As a result, x-rays that pass through the edges 12, 14 have a higher energy than those that pass through the interior and may pass right through any contaminants in those areas without detecting them. Therefore, the x-ray device may detect contaminants in the thickest part of the product's cross sectional area, but could fail to detect contaminants present in the edge portions.

Contaminants which may be present in a round, spherical or cylindrical product, could be inherent, such as bone or cartilage. Alternatively, a contaminant may be extrinsic, foreign matter, such as a metal (like a metal clip), wood or plastic contaminant. A product containing such a contaminant may be discarded, or the contaminant may be removed and the product reprocessed. An apparatus and method for contaminant detection is described. More particularly, the apparatus and method allow for improved contaminant detection rates in irregularly-shaped products, especially those having a circular cross-sectional shape. The apparatus includes a contaminant detection means or device, or contaminant detector. The contaminant detector may be an x-ray source, a source of ultrasound energy, a source of near infrared energy, or a source of electron beam energy, for examples. Other forms of electromagnetic energy that may be provided by the contaminant detection device are ultraviolet light, visible light, near infrared, microwave, gamma radiation and electron beam irradiation. In one embodiment, the contaminant detection device is x-ray equipment, such as an x-ray machine.

The contaminant detection device may be coupled to a conveyor. In one embodiment, the conveyor comprises a conveyor belt operably connected to a contour filler. When a round, spherical or cylindrical product enters a defined zone relative to the contaminant detector, the system activates a contaminant detection procedure that may include activation of the contour filler. The contour filler is any suitable device comprising an energy absorbent material that creates a contour which operates to fill in the cross sectional area of the less thick portion of the product, to mimic a more uniform cross section, thereby optimizing the effectiveness of the contaminant detection device in detecting contaminants in the product. The energy absorbent material is selected based on the energy of the contaminant detector. For example, where the contaminant detector is x-ray equipment, the contour filler comprises x-ray absorbent material. More particularly, the contour filler operates to artificially increase the product's mass around its outside edge, to increase contaminant detection rates whenever contaminants are present at the outside edge of round, spherical or cylindrical products.

In accordance with one embodiment of the invention, a contour filler is provided to hold and/or partially surround a round, spherical or cylindrical product as the product is conveyed through x-ray equipment. The contour filler is any suitable device comprising an x-ray absorbent material that creates a contour, which, essentially, operates to add to or fill in the cross sectional area of the less thick portions of the cross section of round, spherical or cylindrical products. The resultant shape of the contour filler and product may mimic a square or rectangular cross section, for examples. Other shapes are also contemplated, however. The purpose is to fill in around the thinner portions or edges of the circular, spherical or cylindrical product to eliminate or minimize thin cross-sections. In particular, a square or rectangular shape may optimize the effectiveness of the x-ray equipment in detecting contaminants throughout the entire round, spherical or cylindrical product.

In one embodiment, the invention provides a contour filler constructed and arranged to wrap around a round, spherical, or cylindrical product such as a ground beef chub or a sausage chub as the product is conveyed through an x-ray machine. The contour filler may be made of any suitable material, non-limiting examples of which are described herein below.

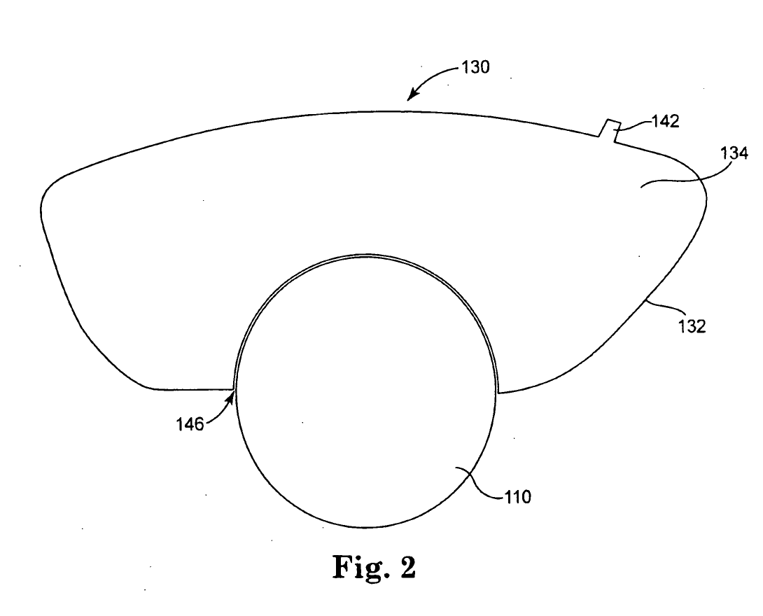

Referring to FIG. 2, in one embodiment, the contour filler 130 comprises an exterior portion (or casing) 132 made of a compliant material, with a fluid material 134 held therein. For example, in the embodiment shown, the contour filler 130 may comprise a compliant exterior portion 132 of a plastic, rubber or elastomer, with a gel or liquid filling material 134 stored therein. The exterior 132 may be, for example, a bag or bladder made of an elastomeric material, the bag, as shown, having a valve 142, and filled with a filling material 134 such as foam, water, gel or silicone, etc. The bag 132 may include a recessed area 146 to accommodate, hold or secure the round, spherical or cylindrical product 110. In use, and as shown, the product 110 is encircled or held in the recessed area 146 by the contour filler 130, and as the product 110 is conveyed through a contaminant detection device. The contaminant detection device may be selected from the group consisting of an x-ray source, a source of ultrasound energy, a source of near infrared energy, and a source of electron beam energy. Other forms of electromagnetic energy that may be provided by a contaminant detection device are ultraviolet light, visible light, near infrared, microwave, gamma radiation and electron beam irradiation. In one embodiment, the contaminant detection device is an x-ray machine.

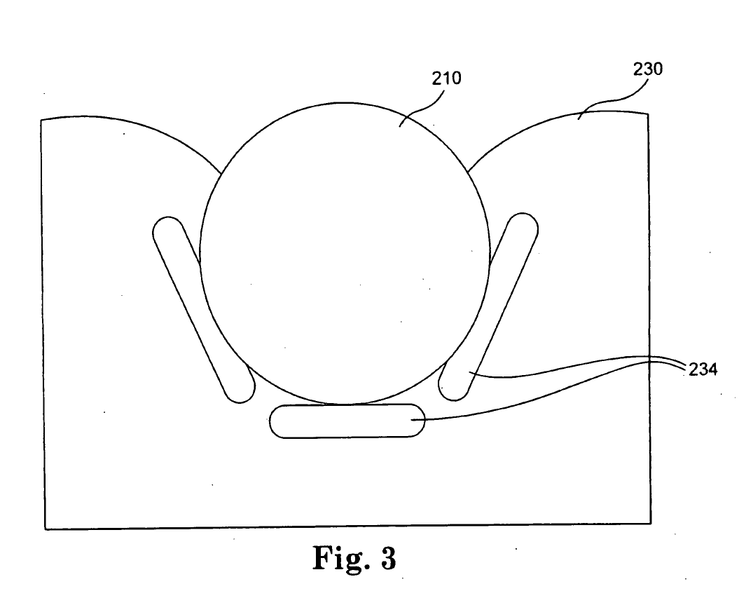

Referring to FIG. 3, in an alternative embodiment, a conveyor 230 may act as a contour filler. As shown, the conveyor 230 may include a U-shaped semi-hemispherical conveyor 234 constructed and arranged to wrap around at least a portion of a round, spherical or cylindrical product 210, and convey the product 210 through a contaminant detection device. The product 210 may rest in the conveyor 230. The conveyor 230 may include a belt 234 that may be segmented, as shown, to accommodate the product 210. The conveyor 230 and/or belt 234 may be made of any suitable compliant material. The conveyor 230 may also comprise any shape that accommodates the shape of a product that is desired, and may fill in around the product in order to allow a means for detecting contamination to detect through all portions of the product, including edge portions. The conveyor 230 may move the product 210 through any type of contaminant detection device, such as those described above.

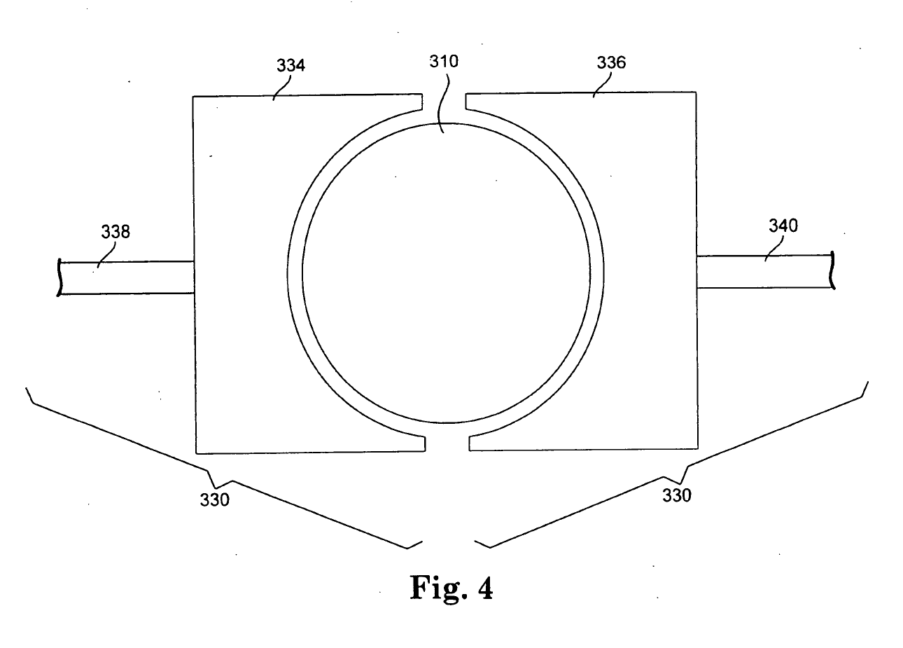

Referring to FIG. 4, in an alternative embodiment, a contour filler 330 may comprise a semi-hemispherical guide-rail assembly 330. The guide rail assembly 330 may comprise a first guide rail 334 and a second guide rail 336, each having a lateral adjustable arm 338, 340. The first and second guide rails 334, 336, are adjustable as to their position by the lateral arms 338, 340. The guide rails 334, 336 are configured to fit around a, round, spherical or cylindrical product 310 and, in use, are extended towards each other in order to close around or surround the product 310. The guide rail assembly 330 holds the product 310 as the product 310 is conveyed through a contaminant detection device. The guide rails 334, 336 may be made of any suitable material, non-limiting examples of which include ultra high molecular weight (UMHW) plastic, such as UMHW polyethylene, and TIVAR®.

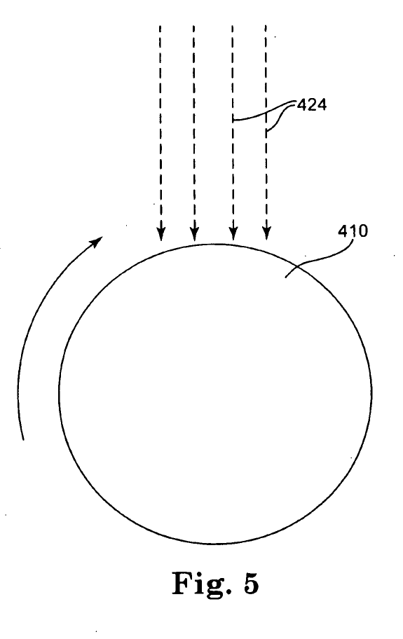

Referring to FIG. 5, an exemplary method for the enhancement of contaminant detection in round, spherical, or cylindrical products is shown. In such a method, the product 410 is rotated, such as shown by the arrow, as product 410 passes through, for example, x-rays 424. Any suitable means for rotating the product 410 along its length are contemplated. One means for rotating the product may involve having a contour filler or conveyor belt (neither shown) comprising a means for retaining and rotating the round, spherical or cylindrical product as it passes through a contaminant detection device. Constant rotation of the round, spherical or cylindrical product 410 positions the product 410 to ensure detection of any contaminants located in the product, including the edge portions. The energy beam from the contaminant detector is generally straight and directional, and the rotation of the product prevents the product from being irradiated in only one position. The product 410 is rotated so that a contaminant located on the periphery of the product will eventually be rotated to a more interior location that would make it detectable by the contaminant detector.

The contaminant detector utilized in the method may also be selected from the group consisting of an x-ray source, a source of ultrasound energy, a source of near infrared energy, and a source of electron beam energy, for examples. Other forms of electromagnetic energy that may be provided by the contaminant detector are ultraviolet light, visible light, near infrared, microwave, gamma radiation and electron beam irradiation.

In one embodiment of the inventive method, the product may be a ground beef chub. As the ground beef chub is rotated, it is exposed to x-ray energy, which allows detection of contaminants in at least one of the rotational positions.

One aspect is a method for detecting contaminants in a product having an irregular shape. The method includes providing a round, spherical or cylindrical object, and providing a contaminant detection device operable within a predetermined zone of operation, and constructed and arranged to detect contaminants in a round, spherical, or cylindrical object within the zone of operation. The method further includes automatically initiating a contaminant detection process upon detecting the object. In one embodiment, the contaminant detection process includes a contour filling procedure comprising providing a contour filler constructed and arranged to retain and position the object within the zone of operation. In one embodiment, the contaminant detection device has a uniform cross sectional area, and the contour filler positions the object in the zone of operation such that the object fills a first portion of the cross sectional area and the contour filler operates to fill a second remaining portion of the cross sectional area of the zone of operation of the contaminant detection device, in order to enhance contaminant detection by the device. In an alternative embodiment, the contaminant detection method includes providing means for rotating the object relative to the contaminant detection device.

One skilled in the art will recognize any combination of a variety of manufactures of these components could be used.

For example, in one embodiment, the method comprises providing a contaminant detection system including an x-ray source operably connected to a conveyor belt operably connected to a contour filler. When a round, spherical, or cylindrical product enters a defined zone of operation of the x-ray source, the system activates a contaminant detection procedure, including activation of a contour filler.

Some commercially available x-ray machines that may be used include those from Smiths Detection Product Inspection, 3202 Regal Drive, Alcoa, Tenn. 37701, U.S.A., (http://www.smithsdetectionpid.com/packaged_product.php). Other such commercially available machines are made by FOSS, 7682 Executive Drive, Eden Prairie, Minn. 55344, U.S.A., (http://www.foss.dk/Solutions/ProductsDirect/MeatMaster/DualSource.aspx). The commercially available contaminant detectors have the limitations on detection set forth above. These limitations have necessitated addition of the contour filler to make these machines capable of detecting contaminants in all portions of irregular shaped products. Contour filling is needed to make x-ray more applicable in the relevant industry.

In summary, the disclosure is directed to an apparatus and method for contaminant detection in a product having an irregular shape. More particularly, the detection of contaminants is in a food product having a round, spherical or cylindrical shape.

In accordance with the disclosure, an apparatus for detecting contaminants in a food product having an irregular shape is provided. The apparatus includes a contaminant detection device constructed and arranged to detect contaminants in a food product when the product enters a zone of operation of the device. The apparatus further includes a conveyor belt operably connected to the contaminant detection device for conveying the food product to the zone of operation. A contour filler is electrically coupled to the conveyor belt and the contaminant detection device. The contour filler is constructed and arranged to retain and position the food product within the zone of operation.

In one embodiment, the zone of operation of the contaminant detection device has a generally uniform cross sectional area and the contour filler is constructed and arranged to position the food product in the zone of operation, such that the food product fills a first portion of the cross sectional area of the zone of operation, and the contour filler fills a second remaining portion of the cross sectional area of the zone of operation.

In another embodiment, the apparatus or contour filler comprises an exterior portion (or casing) made of a soft compliant material filled with a fluid. The exterior may further comprise a recessed portion shaped to accommodate the food product. The exterior may be made of a material selected from the group consisting of a plastic, rubber or elastomer. The exterior is filled with a suitable fluid material which may be selected from the group consisting of a liquid, gel or soft material. In a further embodiment, the contour filler comprises a bag or bladder made of an elastomeric material, encasing a filling material selected from the group consisting of water, foam or silicone, for examples.

In an alternative embodiment, the contour filler comprises a flexible semi-hemispherical conveyor belt. The semi-hemispherical conveyor belt may comprise a plurality of flexible segments constructed and arranged to retain the food product.

In an alternative embodiment, the contour filler comprises a pair of guide rails constructed and arranged to hold the food product in an interior portion thereof, the pair further comprising a first adjustable hemispherical guide rail and a second adjustable semi-hemispherical guide rail. The first and second guide rails each further comprise a lateral adjustable arm, and are adjustable as to their position by the lateral arms. The guide rail assembly may be made of any suitable material, for example, a material selected from the group consisting of UMHW polyethylene, and TIVAR®.

In an alternative embodiment, the contour filler comprises a retaining means and a means for rotating the food product within the zone of operation in an axis perpendicular thereto.

Also described is a method for detecting contaminants in a product having an irregular shape. A round, spherical or cylindrical object is provided. A contaminant detection device is provided. The device is operable within a predetermined zone of operation having a uniform cross sectional area, the device being constructed and arranged to detect contaminants in a round, spherical, or cylindrical object within its zone of operation. A contaminant detection process is initiated upon detection of the object within the zone of operation. A contour filling procedure to enhance contaminant detection by the contaminant detection device is employed.

In one embodiment, the contour filling process includes providing a contour filler constructed and arranged to retain and position the object within the zone of operation, such that the object fills a first portion of the cross sectional area and the contour filler operates to fill a second remaining portion of the cross sectional area of the zone of operation of the contaminant detection device.

In an alternative embodiment, the method includes a contour filling procedure of providing means for rotating the object relative to the contaminant detection device.

While the invention has been described in detail herein in accordance with certain preferred embodiments thereof, many modifications and changes therein may be effected by those skilled in the art without materially departing from the novel teachings and advantages of this invention. Accordingly, all such modifications are intended to be included within the scope of this invention as defined in the following claims and, therefore, it is our intent to be limited only by the scope of the appending claims and not by way of the details and instrumentalities describing the embodiments shown herein.

While different embodiments of the invention have been described in detail herein, it will be appreciated by those skilled in the art that various modifications and alternatives to embodiments could be developed in light of the overall teachings of the disclosure. Accordingly, the particular arrangements are illustrative only and are not limiting as to the scope of the invention, which is to be given the full breadth of any and all equivalents thereof.

What is claimed is:

1. An apparatus for detecting contaminants in a food product having an irregular shape, the apparatus comprising:
a) a contaminant detection device constructed and arranged to detect contaminants in a food product when the food product enters a predetermined zone of operation relative to the contaminant detection device;
b) a conveyor belt electrically coupled to the contaminant detection device for conveying the food product to the zone of operation; and
c) a contour filler electrically coupled to the conveyor belt and the contaminant detection device, the contour filler being constructed and arranged to retain and position the food product within the zone of operation.

2. The apparatus of claim 1, wherein:
a) the zone of operation of the contaminant detection device has a uniform cross sectional area; and
b) the contour filler is constructed and arranged to position the food product in the zone of operation, such that the food product fills a first portion of the cross sectional area of the zone of operation, and the contour filler fills a second remaining portion of the cross sectional area of the zone of operation.

3. The apparatus of claim 1, wherein the contaminant detection device is selected from the group consisting of an x-ray source, a source of ultrasound energy, a source of near infrared energy, a source of electron beam energy, a source of ultraviolet light, a source of visible light, a source of microwave, and a source of gamma radiation.

4. The apparatus of claim 1, wherein the contaminant detection device is an x-ray source, and zone of operation corresponds to an x-ray beam emitted there from.

5. The apparatus of claim 1, wherein the contour filler comprises an exterior portion made of a soft, compliant material, filled with a fluid.

6. The apparatus of claim 5, wherein the exterior of the contour filler includes a recessed portion shaped to accommodate the food product.

7. The apparatus of claim 5, wherein the exterior portion is made of a material selected from the group consisting of a plastic, rubber or elastomeric material, and the contour filler is filled with a fluid material selected from the group consisting of a liquid, gel or soft material.

8. The apparatus of claim 7, wherein the contour filler comprises a bag or bladder made of an elastomeric material, encasing a filling material selected from the group consisting of water, foam or silicone.

9. The apparatus of claim 1, wherein the contour filler comprises a flexible semi-hemispherical conveyor belt.

10. The apparatus of claim 9, wherein the semi-hemispherical conveyor bell comprises a plurality of flexible segments constructed and arranged to retain the food product.

11. The apparatus of claim 1, wherein the contour filler comprises a pair of guide rails constructed and arranged to hold the food product in an interior portion thereof, the pair further comprising a first adjustable semi-hemispherical guide rail and a second adjustable semi-hemispherical guide rail.

12. The apparatus of claim 11, wherein the first and second guide rails each further comprise a lateral adjustable arm, and are adjustable as to their position by the lateral arms.

13. The apparatus of claim 11, wherein the guide rails comprise a material selected from the group consisting of UMHW polyethylene, and TIVAR®.

14. The apparatus of claim 1, wherein the contour filler comprises a retaining means and a means for rotating the food product within the zone of operation in an axis perpendicular thereto.

15. A method for detecting contaminants in a product having an irregular shape, the method comprising the steps of:
a) providing a round, spherical or cylindrical object;
b) providing a contaminant detection device operable within a predetermined zone of operation having a uniform cross sectional area, the device being constructed and arranged to detect contaminants in a round, spherical, or cylindrical object within the zone of operation;
c) initiating a contaminant detection process upon detecting the object within the zone of operation; and
d) initiating a contour filling procedure to enhance contaminant detection by the contaminant detection device.

16. The method of claim 15, wherein the contour filling procedure includes the step of providing a contour filler constructed and arranged to retain and position the object within the zone of operation, such that the object fills a first portion of the cross sectional area of the zone of operation and the contour filler operates to fill a second remaining portion of the cross sectional area of the zone of operation of the contaminant detection device.

17. The method of claim 15, wherein the contour filling procedure includes the step of providing means for rotating the object relative to the contaminant detection device.

18. The method of claim 15, wherein the contaminant detection device is selected from the group consisting of an x-ray source, a source of ultrasound energy, a source of near infrared energy, a source of electron beam energy, a source of ultraviolet light, a source of visible light, a source of microwave, and a source of gamma radiation.

19. An apparatus for enhancement of contaminant detection in a product having an irregular shape using a contaminant detection device, the apparatus comprising a contour filler comprising a recessed portion to accommodate the irregular shape of the product, wherein when the product is in the recessed portion, together the contour filler and product provide a cross sectional area in which contaminants may be identified by the contaminant detection device.

20. The apparatus of claim 19, wherein the cross sectional area of the contour filler and product together is substantially uniform.

21. The apparatus of claim 19, wherein the contour filler comprises two portions that combine to provide the recessed portion.

22. The apparatus of claim 19, wherein the contaminant detection device is selected from the group consisting of an x-ray source, a source of ultrasound energy, a source of near infrared energy, a source of electron beam energy, a source of ultraviolet light, a source of visible light, a source of microwave, and a source of gamma radiation.

23. The apparatus of claim 19, wherein the contour filler comprises an exterior portion made of a soft, compliant material, filled with a fluid.

24. The apparatus of claim 19, wherein the contour filler comprises a conveyor belt.

25. The apparatus of claim 19, wherein the cross sectional shape is selected from the group consisting of a square and a rectangle.
